(19) United States
(12) Patent Application Publication
(10) Pub. No.: US 2009/0306080 A1
(43) Pub. Date: Dec. 10, 2009

(54) VETERINARY FORMULATION

(76) Inventors: Majid Hameed Abdul RAZZAK, Auckland (NZ); Vijay Madhoo, Auckland (NZ)

Correspondence Address: Judy Jarecki-Black, Ph.D.;Merial Ltd., 3239 Satellite Blvd., Duluth, GA, 30096-4640, US

(21) Appl. No.: 12/484,950

(22) Filed: Jun. 15, 2009

Related U.S. Application Data
(63) Continuation-in-part of application No. PCT/NZ2007/000363, filed on Dec. 14, 2007.

(30) Foreign Application Priority Data
Dec. 15, 2006 (NZ) 552096

(51) Int. Cl. A61K 31/53; A61P 33/14

(52) U.S. Cl. 514/245

(57) ABSTRACT

The present invention relates to a liquid veterinary formulation comprising a stable solution of cyromazine, or a pharmaceutically acceptable salt thereof, in a solvent system which may comprise water and one or more polyethylene glycols. A liquid concentrate is also described in which the cyromazine, or a pharmaceutically acceptable salt thereof, may be dissolved in a liquid PEG and is capable of being diluted with water before use.

### INCORPORATION BY REFERENCE

This application is a continuation-in-part of international patent application Serial No. PCT/NZ2007/000363 filed Dec. 14, 2007, which published as PCT Publication No. WO 2008/072987 on Jun. 19, 2008, which claims priority from New Zealand Patent Application No. 552096 filed Dec. 15, 2006.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to formulations of the insecticide 2-cyclopropyl-amino-4,6-diamino-s-triazine (common name cyromazine).

### BACKGROUND OF THE INVENTION

Sheep and other domesticated livestock animals, such as cattle, horses, goats, swine and poultry are subject to infestation by a wide range of ectoparasites. Ectoparasites are those parasites which live on the outer surface of the host but still draw nutrients from the host such as fleas, lice, blow fly, ticks, mites, head fly, keds and sheep scab. Of particular importance is the sheep blow fly, whose larvae constitutes a parasite that can cause significant suffering and loss of production in infected sheep. At certain times of the year when blow flies are active, the adult blow fly will lay eggs on sheep. When the eggs hatch the larval stage will then feed on the flesh of the infected sheep, causing what is known as blow fly strike or sheep myiases.

Over the years a wide variety of treatments have been used to both treat and prevent infestation by blow fly. These have included organophosphate treatments and synthetic pyrethroid treatments that act via contact with or ingestion by the parasite. Another class of chemicals used for blow fly treatment or prevention are the Insect Growth Regulators (IGRs). This class of compounds is made up of two sub-classes—juvenile hormone mimics and chitin synthesis inhibitors (CSIs).

Hydroprene and methoprene are examples of juvenile hormone mimics. These pesticides mimic the juvenile hormone produced in the insect brain, which forces the insect to remain in a juvenile state. By contrast, CSIs such as triflumuron, lufenuron, and diflubenzuron inhibit the production of chitin, a major component of the insect exoskeleton. Insects treated with CSIs are unable to synthesize new cuticle and are therefore unable to successfully moult into the next stage of their life cycle.

Cyromazine (2-cyclopropylamino-4,6-diamino-s-triazine) and salts thereof are commercially available from a variety of sources. Cyromazine can be used in particular for controlling a variety of insect pests, including hygiene pests and animal ectoparasites of the order Diptera, such as, for example, flies, gnats and mosquitoes. In general use it is important that the formulations are stable for a reasonable period of time and are able to withstand a variety of climatic and temperature conditions.

One approach has been to produce certain salts of cyromazine in situ during the preparation of formulations to aid solubility. Cyromazine has the ability to form the mono and di salts with various organic and inorganic acids because it exhibits weak basic characteristics.

Despite this ability to form salts many of the salts produced are also poorly soluble in water with only a few having solubilities in excess of 100 g/L in water at 20° C. Some examples exhibiting improved solubility are the acetate; lactate; sulphate and tartrate salts of cyromazine. However, when subjected to temperatures below 20° C., their water solubility is reduced further. This reduced solubility may result in the formation of crystals which tend not to re-dissolve when the formulation returns to an ambient temperature.

Thus, there is a need to provide a highly soluble formulation of cyromazine that is stable and avoids the problems of poor aqueous solubility and crystallisation.

Citation or identification of any document in this application is not an admission that such document is available as prior art to the present invention.

### SUMMARY OF THE INVENTION

It is therefore an object of the present invention to provide a stable aqueous based formulation of cyromazine which attempts to overcome at least some of the disadvantages and limitations of the known art or which at least provides the public with a useful choice.

In a first aspect the invention relates to a liquid veterinary formulation comprising a stable solution of cyromazine, or pharmaceutically acceptable salts thereof, in a solvent system containing water and a sufficient amount of one or more of the polyethylene glycols (PEGs) (either a single grade or a combination of two or more different grades) to keep the cyromazine in solution at ambient temperature.

In a second aspect the invention relates to a topical liquid veterinary formulation comprising a stable solution of cyromazine, or pharmaceutically acceptable salts thereof, in a solvent system containing water and one or more of the polyethylene glycols (either a single grade or a combination of two or more different grades), wherein the total amount of polyethylene glycol in the formulation is at least about 30% v/v.

In a further aspect the invention relates to a method of treating, controlling or preventing ectoparasites in an animal by topically administering to said animal a liquid veterinary formulation comprising a stable solution of cyromazine, or pharmaceutically acceptable salts thereof, in a solvent system containing water and a sufficient amount of one or more of the polyethylene glycols (either a single grade or a combination of two or more different grades) to keep the cyromazine in solution at ambient temperature.

It is noted that in this disclosure and particularly in the claims and/or paragraphs, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

These and other embodiments are disclosed or are obvious from and encompassed by, the following Detailed Description.

### DETAILED DESCRIPTION

The following description will describe the invention in relation to advantageous embodiments thereof. The invention is in no way limited to these advantageous embodiments as they are purely to exemplify the invention and the invention is intended to include possible variations and modifications as would be readily apparent to a person skilled in the art without departing from the scope of the invention.

The invention relates to a liquid veterinary formulation comprising a stable solution of cyromazine, or pharmaceutically acceptable salts thereof, in a solvent system containing water and a sufficient amount of one or more of the polyethylene glycols (either a single grade or a combination of two or more different grades) to keep the cyromazine, or pharmaceutically acceptable salts thereof, in solution at ambient temperature. The invention further relates to a method of treating, controlling or preventing ecto-parasites in an animal by administering to said animal such a formulation. The formulation has been found to be particularly effective in the treatment, control or prevention of blow fly strike, which commonly occurs in sheep.

The basis of this invention is the surprising discovery that by formulating cyromazine in solution with water and one or more of the polyethylene glycols, a stable formulation can be produced, with a significant reduction in the possibility of crystallisation of the active ingredient. The particular combination of water and polyethylene glycol therefore enhances the stability of the cyromazine.

In reaching this solution, the Applicant conducted a series of formulation studies as described hereafter.

The Applicant tested a number of acid combinations to attempt to overcome the crystallisation issues being experienced in the formulation of an aqueous based cyromazine formulation. Although some success was achieved, the problem of crystallisation remained, especially at low temperature.

The solubility of cyromazine in a range of organic solvents including solvents which are commonly used in topical spray-on formulations is shown in the table below. This shows that the solubility of cyromazine in these particular solvents was generally too low to be of practical benefit to the Applicant's project:

| Solvent | Solubility (g/kg) at 20° C. |
|---|---|
| Water | 1.3 (pH 7) |
| Acetone | 1.7 |
| Hexane | 0.0002 |
| Isopropyl Alcohol | 2.5 |
| Methanol | 22 |
| Methylene Chloride | 0.25 |
| n-Octanol | 1.2 |
| Toluene | 0.015 |
| DGBE | 6 |
| NMP | Insoluble |
| Propylene Glycol | Insoluble |

Within the context of the testing programme the Applicant decided to test the solubility of cyromazine in the polyethylene glycols class of compounds.

Polyethylene glycols (PEGs) are a family of water-soluble linear polymers formed by the additional reaction of ethylene oxide (EO) with monoethylene glycol or diethylene glycol. The generalised formula for polyethylene glycol is: H(OCH₂CH₂)ₙOH, where n is the average number of repeating EO groups. There are many grades of PEGs which are represented by their average molecular weight. For example, PEG 400 consists of a distribution of polymers of varying molecular weights with an average of 400, which corresponds to an approximate number of repeating EO groups (n) of nine (9). Polyethylene glycols are commercially available in average molecular weight ranging from 200 to 35000. Depending on their average molecular weights, the PEGs may be liquid or solid under standard conditions. For example, PEG 200, PEG 300, PEG 400, and PEG 600 are in liquid form at room temperature. PEG 1000, PEG 1500 are in semi solid form at room temperature and PEG 2000 to PEG 35000 are in solid form at room temperature.

Surprisingly, it was found that cyromazine demonstrated a very high level of solubility in the liquid polyethylene glycols, and in the case of PEG 200, up to 180 g/L of cyromazine could be dissolved in this solvent. In the following examples the formulations containing liquid PEGs are shown as % v/v (e.g. for PEG 200 and 400) but for the slid PEGS the formulations are shown as % w/v (e.g. the examples containing PEG 8000 or 35000). The results of the solubility studies are shown in the following table:

| PEG | Temperature | Cyromazine g/100 ml | Observations |
|---|---|---|---|
| 200 | Room | 18 | Solution clear |
| 200 | 70° C. | 18 | Solution clear |
| 400 | Room | 10 | Solution clear |
| 400 | 70° C. | 10 | Solution clear |

Given the high level of solubility of cyromazine in PEG 200 and PEG 400 as shown in table 2 above, the Applicant decided to evaluate the solubility of cyromazine in various mixtures of PEG and water, in order to obtain an aqueous based formulation.

In a first aspect the invention may relate to a liquid veterinary formulation comprising a stable solution of cyromazine in a solvent system containing water and a sufficient amount of one or more of the polyethylene glycols (PEGs) (either a single grade or a combination of two or more different grades) to keep the cyromazine in solution at ambient temperature.

Advantageously, the majority of the polyethylene glycols used in the formulation have an average molecular weight in the range from about 200 to about 35000.

Advantageously, each polyethylene glycol is completely or predominantly a liquid at ambient temperature. Examples of liquid PEGs include, but are not limited to PEG 200 and PEG 400. In other embodiments, PEG 300 or PEG 600 may be used.

Alternatively, each polyethylene glycol may be a solid at ambient temperature. Examples of solid PEGs include, but are not limited to PEG 1000, PEG 6000, PEG 8000 and up to and including PEG 35000. However if a solid PEG is used in the formulation it may be necessary to heat the solid PEG until it is molten to allow the cyromazine to be dissolved or dispersed therein, and then combine the molten PEG and cyromazine with water to create a stable solution. Alternatively, if the solid PEG is mixed with the water first and then the cyromazine is added it may not be necessary to heat the solid PEG.

Advantageously, the cyromazine, or a pharmaceutically acceptable salt thereof, is present in an amount of from about 0.1 to about 20% w/v, with a range of about 1 to about 10% w/v being advantageous, with about 5 to about 7% w/v being especially advantageous. A most especially advantageous amount of cyromazine is about 6% w/v.

If the cyromazine, or a pharmaceutically acceptable salt thereof, is present in an amount of about 6% w/v or more, then advantageously the total amount of PEG or PEGs in the formulation is from about 30% v/v to about 99% v/v, and more advantageously the total amount of PEG or PEGs in the formulation is from about 40% v/v to about 90% v/v.

If the cyromazine, or a pharmaceutically acceptable salt thereof, is present in an amount of about 6% w/v or more, then advantageously, the formulation contains at least about 40% v/v of PEG if the formulation is prepared without heating. If less than about 40% v/v of PEG is used, it may be necessary to heat the dispersion to dissolve the cyromazine.

In a second aspect the invention may broadly be said to relate to a topical liquid veterinary formulation comprising a stable solution of cyromazine, or pharmaceutically acceptable salts thereof, in a solvent system containing water and one or more of the polyethylene glycols (either a single grade or a combination of two or more different grades), wherein the total amount of polyethylene glycol in the formulation is at least about 30% v/v.

The formulations of the present invention may further comprise at least one additional active ingredient, such as additional parasiticide or an anti-parasitic agent, which includes, but is not limited to other insect growth regulator (juvenile hormone mimics or CSI), avermectin, milbemycin, morantel, pyrantel, febrantel, benzimidizole, such as thiabendazole or cambendazole, nodulisporic acid derivative, 1-N-arylpyrazole and any combinations thereof. It is known in the art that it is sometimes possible to combine various parasiticides in order to broaden the antiparasitical spectrum.

The term “acid” contemplates all pharmaceutically or veterinary acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically or veterinary acceptable aliphatic, alicyclic and aromatic carboxylic acids, dicarboxylic acids tricarboxylic acids and fatty acids. Preferred acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, fumaric acid, acetic acid, propionic acid, isopropionic acid, valeric acid, α-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tataric acid and maleic acid. An example of a tricarboxylic acid is citric acid. Fatty acids include all pharmaceutically or veterinary acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

In one embodiment, the present invention includes pharmaceutically acceptable salts of cyromazine, including mono and di-salts with suitable organic or inorganic acids. Suitable salts of cyromazine include, but are not limited to, acetate; lactate; sulphate and tartrate salts of cyromazine.

The formulations of the present invention may include further excipients such as stabilizers, colouring agents, preservatives, buffers, thickeners, spreading agents, wetting agents, surfactants, crystallization inhibitors and the like. The formulations of the present invention may include further excipients such as pharmaceutically acceptable lubricant or solubilizer selected from the group consisting of neutral oils of esters of saturated coconut and palm oils, corn oil, mineral oils, castor oil, hydrogenated castor oil, oxygenated castor oil, fractionated coconut oil, peanut oil, sesame oil, a surfactant and mixtures thereof. The presence of a solubilizer is to enhance the solubility or bioavailability of the pharmaceutically active material. A solubilizer may be used as a wetting agent for the pharmaceutically active material, or can also be used as a surfactant or can also be used as a lubricant. Solubilizers can be water or oil based. Non-limiting examples of solubilizers include neutral oils of esters of saturated coconut and palm oils (Miglyol® neutral oils, e.g. Miglyol® 810, 812, 818, 829, and 840, castor oil based surfactants (e.g. Sufactol® 318 (PEG-5) or Surfactol® 365 (PEG-40), corn oils, mineral oils, peanut oil, sesame oil, fractionated oils and surfactols.

The surfactant can be anionic, cationic, nonionic or amphoteric. Non limiting examples of anionic surfactants are alkaline stearates, such as sodium, potassium or ammonium stearate; calcium stearate or triethanolamine stearate; sodium abietate; alkyl sulphates, such as sodium lauryl sulphate and sodium cetyl sulphate; sodium dodecylbenzenesulphonate or sodium dioctyl sulphosuccinate; or fatty acids, such as those derived from coconut oil. Example of cationic surfactants, such as water-soluble quaternary ammonium salts of formula N⁺R′R″R′″R″″Y⁻, in which the R groups are identical or different optionally hydroxylated hydrocarbon groups and Y⁻ is an anion of a strong acid, such as halide, sulphate and sulphonate anions; or cetyltrimethylammonium bromide and the like.

Non limiting examples of non-ionic surfactants are polyoxyethylenated esters of sorbitan, in particular polysorbate 80 (Tween® 80), or polyoxyethylenated alkyl ethers; polyethylene glycol stearate, polyoxyethylenated derivatives of castor oil, such as PEG-5 to PEG-100 hydrogenated castor oil and in particular PEG-40 hydrogenated castor oil, polyglycerol esters, polyoxyethylenated fatty alcohols, polyoxyethylenated fatty acids or copolymers of ethylene oxide and of propylene oxide and glyceryl monostearate.

Non-limiting examples of amphoteric surfactants are substituted lauryl compounds of betaine.

Crystallization inhibitors which can be used in topical formulations in the present invention include, but are not limited to:
polyvinylpyrrolidone (PVP), polyvinyl alcohols, polyvidone, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others,
anionic surfactants, such as alkaline stearates, in particular sodium, potassium or ammonium stearate; calcium stearate or triethanolamine stearate; sodium abietate; alkyl sulphates, in particular sodium lauryl sulphate and sodium cetyl sulphate; sodium dodecylbenzenesulphonate or sodium dioctyl sulphosuccinate; or fatty acids, in particular those derived from coconut oil,
cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulphate and sulphonate anions; cetyltrimethylammonium bromide is one of the cationic surfactants which can be used,
amine salts of formula N⁺R′R″R′″, in which the R radicals are identical or different optionally hydroxylated hydrocarbon radicals; octadecylamine hydrochloride is one of the cationic surfactants which can be used,
non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide,
amphoteric surfactants, such as substituted lauryl compounds of betaine,
or advantageously a mixture of at least two of the compounds listed above. In a particularly advantageous embodiment, a crystallization inhibitor pair will be used.

Such pairs include, for example, the combination of a film-forming agent of polymeric type and of a surface-active agent. These agents will be selected in particular from the compounds mentioned above as crystallization inhibitor.

Particularly advantageous film-forming agents of polymeric type include:
the various grades of polyvinylpyrrolidone (PVP),
polyvinyl alcohols, and
copolymers of vinyl acetate and of vinylpyrrolidone.

Especially advantageous surface-active agents, include those made of non-ionic surfactants, advantageously polyoxyethylenated esters of sorbitan and in particular the various grades of polysorbate, for example Polysorbate 80.

The film-forming agent and the surface-active agent can in particular be incorporated in similar or identical amounts within the limit of the total amounts of crystallization inhibitor mentioned elsewhere.

The pair thus constituted secures, in a noteworthy way, the objectives of absence of crystallization on the coat and of maintenance of the cosmetic appearance of the fur, that is to say without a tendency towards sticking or towards a sticky appearance, despite the high concentration of active material.

The crystallization inhibitor can in particular be present in a proportion of about 1 to about 20% (W/V), advantageously of about 2 or about 5 to about 15%.

Additionally, the inventive formulations may contain other inert ingredients such as antioxidants, pH stabilizers, preservatives, or colorants. These compounds are well known in the formulation art and do not change the novel and basic characteristics of the invention. Antioxidant or pH stabilizers include alpha tocopherol, ascorbic acid, ascrobyl palmitate, fumaric acid, malic acid, citric acid, sodium ascorbate, sodium metabisulfate, n-propyl gallate, BHA (butylated hydroxy anisole), BHT (butylated hydroxy toluene) monothioglycerol, sodium phosphate monobasic, sodium phosphate tribasic, and the like, may be added to the present formulation. The antioxidants or pH stabilizers are generally added to the formulation in amounts of from about 0.01 to about 2.0%, based upon total weight of the formulation, with about 0.05 to about 0.5% being especially advantageous BHA.

Preservatives, such as the parabens (methylparaben and/or propylparaben), are suitably used in the formulations in amounts ranging from about 0.01 to about 2.0%, with about 0.05 to about 1.0% being especially advantageous. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Advantageous ranges for these compounds include from about 0.01 to about 5%.

The thickeners contemplated by this invention are well known to a practitioner of this art. Compounds which function as thickeners include, for example, povidone, maltodextrin, polydextrate, EMDEX (dextrates), carboxypolymethylene (Carbomer®), polyethylene glycol and celluloses, such as hydroxypropyl celluloses. An especially advantageous thickener is povidone. Thickeners may be present in amounts of from about 0.1% to about 25%.

Opacifiers may be added to absorb and/or reflect certain light and/or energy of certain wavelengths and may thus enhance the stability of the formulations. Opacifiers include, for example, zinc oxide or titanium dioxide. These compounds are well known to practitioners of this art.

Colorants may be added to the inventive formulations. Colorants contemplated by the present invention are those commonly known in the art. Specific colorants include, but are not limited to dyes, an aluminum lake, caramel, organic dyes, titanium dioxide, colorant based upon iron oxide or a mixture of any of the foregoing. Advantageous ranges include from about 0.001% to about 25%.

Compounds which acidify the formulation are also contemplated. Again, acidifying compounds and their use to lower the pH of a formulation are well known to a practitioner in the art. Examples of such acidifying stabilizers include, but are not limited to compounds selected from the group consisting of ascorbic acid, malic acid, isoascorbic acid, cysteine hydrochloride, cysteine dihydrochloride, citric acid fumaric acid, acetic acid, sorbic acid, glycine hydrochloride, arginine hydrochloride, succinic hydrochloride, succinic acid, tartaric acid, phosphoric acid, hydrochloric acid, glucono-delta-lactone, and the like. Chelating agents may include EDTA, diethanolamine and triethanolamine.

The inventive topical formulations may also contain penetration enhancers, such as dimethylacetamide, Transcutol®, DMSO or dimethyl isorbide, or chelating agents. Penetration enhancers are used in small amounts, amounts that are of such quantity that they will not dissolve the actives.

The formulations of the present invention have been found to be effective for the treatment, control or prevention of ectoparasites, particularly blow fly on sheep. Advantageously, the formulations are designed to be spot-on, pour-on or spray-on formulations for topical administration. The topical formulations may be used to treat a number of ectoparasite infections. The determining of a treatment protocol for an infection of a specific parasite or parasites would be well within the skill level of a practitioner of the veterinary art.

Cyromazine may be administered alone or in a formulation appropriate to the specific use envisaged and to the particular type of animal being treated. A variety of methods can be used for oral administration, including but not limited to capsule, bolus, tablet, liquid, or feed additive formulations. Such formulations are prepared in a conventional manner in accordance with standard veterinary practice. Thus, capsules, boluses or tablets may be prepared or formulated by mixing the active substance with a suitable finely divided diluent or carrier additionally containing a disintegrating agent and/or binder such as starch, lactose, talc, magnesium stearate, gelatin, and the like. Oral drenches are prepared by dissolving or suspending the active ingredient cyromazine in a suitable medium. The term “formulated” also means, for example, preparing cyromazine in the form of a powder, a tablet, a granulate, a capsule, an emulsion, a foam, and other means known in the art. Liquid formulations may be also used to supplement drinking water, or to sprinkle or spread onto feed.

With topical applications including, but limited to spray-on or pour-on applications, aqueous based formulations are preferred. This type of formulation allows an even spread and accurate dosing of the active ingredient around the infected areas of the animal. However, given that cyromazine has poor solubility in water, aqueous based formulations of cyromazine are difficult to formulate. Various attempts have been made to overcome this problem of poor solubility.

The methods of the present invention are particularly useful for control of insect pests of the following families: Muscidae (for example, house flies, stable flies, horn flies, and face flies), Mycetophilidae (for example, fungus gnats), Chloropidae (for example, eye gnats), Culicidae (for example, mosquitoes), Simuliidae (for example, black flies), Tipulidae (for example, crane flies), Calliphoridae (for example, blow flies), Gasterophilidae (for example, bot flies), and Tabanidae (for example, horse flies and deer flies). Accordingly, the present invention provides methods for the control of flies, wherein said flies are selected from the group consisting of stable flies, horn flies, house flies, face flies and a combination of two or more thereof. Cyromazine is a CSI that has achieved widespread use as a means to control blow fly larvae. It is applied to sheep in the form of a dip, pour on or a spray on. In the dip form, animals are completely saturated with the formulation, whereas with the pour on and spray formulation, only those areas of the animal likely to be infected by blow fly larvae are treated.

Administration of the topical formulation may be intermittent in time and may be administered daily, weekly, biweekly, monthly, bimonthly, quarterly, or even for longer durations of time. The time period between treatments depends upon factors such as the parasite(s) being treated, the degree of infestation, the type of mammal or bird and the environment where it resides. It is well within the skill level of the practitioner to determine a specific administration period for a particular situation.

Spot-on formulations may be prepared by dissolving the active ingredients into the pharmaceutically or veterinary acceptable vehicle. Alternatively, the spot-on formulation can be prepared by encapsulation of the active agent to leave a residue of the therapeutic agent on the surface of the animal. These formulations will vary with regard to the weight of the therapeutic agent in the combination depending on the species of host animal to be treated, the severity and type of infection and the body weight of the host. The compounds may be administered continuously, particularly for prophylaxis, by known methods.

Generally, a dose of from about 0.001 to about 10 mg per kg of body weight given as a single dose or in divided doses for a period of from 1 to 5 days will be satisfactory but, of course, there can be instance where higher or lower dosage ranges are indicated and such are within the scope of this invention. It is well within the routine skill of the practitioner to determine a particular dosing regimen for a specific host and parasite. While not wishing to be bound by theory, it is believed that the invention spot-on formulation work by the dose dissolving in the natural oils of the host's skin, fur or feathers. Further as the inventive formulations are not ingested orally, one does not have to be concerned about disguising the taste of the active agents.

The formulations of the present invention may provide for the topical administration of a concentrated solution, suspension, microemulsion or emulsion for intermittent application to a spot on the animal, generally between the two shoulders (for example, solution of spot-on type). The liquid carrier vehicle for topical, for example spot-on, formulations comprises a pharmaceutically or veterinary acceptable organic solvent which may include, but is not limited to acetone, ethyl acetate, methanol, ethanol, isopropanol, benzyl alcohol, dimethylformamide, pyrrolidones, N-methylpyrrolidone, diethylene glycol butyl ether, dipropylene glycol monoethyl ether, diethylene glycol monoethyl ether (Transcutol) and other glycol derivatives, such as propylene glycol, glycol ethers, polyethylene glycols and glycerol, and optionally an organic cosolvent.

The co-solvents are also well known to a practitioner in the formulation art. Advantageous co-solvents are those which is a promoter of drying and include, for example, methanol, absolute ethanol, ethanol, isopropanol (2-propanol) or benzyl alcohol.

The formulation adjuvants discussed above are well known to the practitioner in this art and may be obtained commercially or through known techniques. These concentrated compositions are generally prepared by simple mixing of the constituents as defined above; advantageously, the starting point is to mix the active material in the main solvent and then the other ingredients or adjuvants are added.

In a further aspect the invention relates to a liquid concentrate capable of being diluted with water to make a stable aqueous solution of cyromazine, the concentrate comprising a stable solution of cyromazine in a liquid polyethylene glycol.

Advantageously the concentrate contains PEG 200 or PEG 400.

In a further aspect the invention may broadly be said to relate to a method of treating, controlling or preventing ectoparasites in an animal by topically administering to said animal a liquid veterinary formulation comprising a stable solution of cyromazine in a solvent system containing water and a sufficient amount of one or more of the polyethylene glycols (either a single grade or a combination of two or more different grades) to keep the cyromazine in solution at ambient temperature.

Advantageously, said ectoparasites are blow fly and/or blow fly larvae, and advantageously said animal is a sheep.

Advantageously, said formulation is a pour-on or spray-on formulation.

Advantageously, said formulation is delivered in a band on the infected area or areas of the animal.

Advantageously, said formulation is delivered in a dosage amount of about 10 ml per 10 kg of live bodyweight of said animal.

This invention may also broadly be said to consist in the parts, elements and features referred to or indicated in the specification of the application, individually or collectively, and in any or all combinations of any two or more of said parts, elements or features, and where specific integers are mentioned herein which have known equivalents in the art to which this invention relates, such known equivalents are deemed to be incorporated herein as if individually set forth.

The invention is further described by way of the following non-limiting examples.

### Example 1

### Formulation 1

The following results were obtained when the solubility potential of a formulation containing PEG 400 and water was tested:

| | Deionised | Cyromazine | |
|---|---|---|---|
| PEG 400 (mL) | Water (mL) | g/100 ml | Observations |
| 40 | 60 | 6 | Solution clear |
| 60 | 40 | 12 | Solution clear |
| 80 | 20 | 12 | Solution clear |

### Example 2

### Formulation 2

This formulation comprises cyromazine in PEG 200 (liquid form) and water.

Method

Six (6) gram quantities of cyromazine were weighed and dissolved with stirring in various mixtures of PEG 200 and water (to 100 ml) at 25° C. Heat was applied where necessary.

Results

| | Deionised | |
|---|---|---|
| PEG 200 (mL) | Water (mL) | Observations |
| 50 | 50 | Solution clear |
| 40 | 60 | Solution clear |
| 30 | 70 | Solution clear after heating to ±60° C. |
| 20 | 80 | Solution clear after heating to ±60° C. |
| | | but crystals present after 24 hours |
| 10 | 90 | Solution clear after heating to ±60° C. |
| | | but crystals present after 24 hours |

### Example 3

### Formulation 3

This formulation comprises cyromazine in PEG 400 (liquid form) and water.

Method

Six (6) gram quantities of cyromazine were weighed and dissolved with stirring in various mixtures of PEG 400 and water (to 100 ml) at 25° C. Heat was applied where necessary.

Results

| | Deionised | |
|---|---|---|
| PEG 400 (mL) | Water (mL) | Observations |
| 50 | 50 | Solution clear |
| 40 | 60 | Solution clear |
| 30 | 70 | Solution clear after heating to ±60° C. |
| 20 | 80 | Solution clear after heating to ±60° C. |
| | | but crystals present after 24 hours |
| 10 | 90 | Solution clear after heating to ±60° C. |
| | | but crystals present after 24 hours |

### Example 4

### Formulation 4

This formulation comprises cyromazine in PEG 1000 (semi solid form) and water.

Method

Various quantities of PEG 1000 were weighed into a glass beaker and heated until melted. Six (6) grams of cyromazine was then added to the molten PEG and stirred until a smooth dispersion was obtained. Deionised water was then added with constant stirring. The temperature was maintained at about 65° C.

Results

| PEG 1000 | Deionised Water | Observations |
|---|---|---|
| 40 g | To 100 ml | Solution clear |
| 30 g | To 100 ml | Crystals present after 24 hours |
| 20 g | To 100 ml | Crystals present after 24 hours |

### Example 5

### Formulation 5

This formulation comprises cyromazine in PEG 6000 (solid form) and water.

Method

Various quantities of PEG 6000 were weighed into a glass beaker and heated until melted. Six (6) grams of cyromazine was then added to the molten PEG and stirred until a smooth dispersion was obtained. Deionised water was then added with constant stirring. The temperature was maintained at about 65° C.

Results

| PEG 6000 | Deionised Water | Observations |
|---|---|---|
| 40 g | To 100 ml | Solution clear |
| 30 g | To 100 ml | Crystals present after 24 hours |
| 20 g | To 100 ml | Crystals present after 24 hours |

### Example 6

### Formulation 6

This formulation comprises cyromazine in PEG 8000 (solid form) and water.

Method

Various quantities of PEG 8000 were weighed into a glass beaker and heated until melted. Six (6) grams of cyromazine was then added to the molten PEG and stirred until a smooth dispersion was obtained. Deionised water was then added with constant stirring. The temperature was maintained at about 65° C.

Results

| PEG 8000 | Deionised Water | Observations |
|---|---|---|
| 40 g | To 100 ml | Solution clear |
| 30 g | To 100 ml | Crystals present after 24 hours |
| 20 g | To 100 ml | Crystals present after 24 hours |

### Example 7

### Formulation 7

This formulation comprises cyromazine in PEG 35000 (solid form) and water.

Method

Various quantities of PEG 35000 were weighed into a glass beaker and heated until melted. Between 2 and 3 grams of cyromazine was then added to the molten PEG as shown in the table below and stirred until a smooth dispersion was obtained. Deionised water was then added with constant stirring. The temperature was maintained at about 65° C.

Results

| PEG 35000 | Deionised Water | % w/v cyromazine | Observations |
|---|---|---|---|
| 10 g | To 100 ml | 2 | Solution Clear |
| 20 g | To 100 ml | 2.5 | Solution Clear |
| 30 g | To 100 ml | 3 | Solution Clear |

Conclusions

As can be seen from the results in respect of examples 1 to 3 above, at 25° C., cyromazine in an amount about 6% w/v or about 12% w/v readily dissolves in mixtures of PEG and water when the liquid PEGs are present at a level of at least 40% v/v. When the PEG is present at a level of about 30% v/v, heating is required in order to dissolve the cyromazine.

Further tests were carried out in the production of concentrate solutions of cyromazine

(containing about 6% w/v cyromazine) in PEG 200 or PEG 400, and the subsequent dilution of these concentrates with water. Comparable results were achieved to those shown above, with stable aqueous solutions containing from about 10% water v/v to about 70% water v/v, although if heating is not to be used, then the water level should not exceed about 60% v/v. From the results in respect of examples 4 to 7 above, it can be seen that cyromazine in an amount of about 6% w/v readily dissolves in mixtures of PEG and water, when the PEG is present in an amount of at least 40% v/v. If less than 40% v/v of a solid PEG (PEG 1000 or higher) is used, crystals occur after a period of time. In the case of a liquid PEG (such as PEG 200 and PEG 400) the threshold is about 30% v/v of the liquid PEG (although as noted above heating of the solution was needed to obtain a clear solution).

The Applicant found that if a solid PEG is used in the formulation it may be heated until it is molten to allow the cyromazine to be dissolved or dispersed therein, and then combine the molten PEG and cyromazine with water to create a stable solution. However, although the methods used in the above examples involve mixing the PEG and cyromazine together first and then adding the water, successful formulations can also be made by mixing the water and PEG together first and then adding the cyromazine. The Applicant has found that this method is an easier method of manufacture. If using this method with solid PEGs, it may not be necessary to heat the solid PEG(s).

The examples above relate to formulations containing at least 6% w/v of cyromazine. If a lower concentration or amount of cyromazine is used, then it is possible to obtain a stable solution of cyromazine by including less than about 30% v/v of PEG and more than about 70% v/v water.

Stability Studies

Based on these experiments, the Applicant decided to subject some batches of Formulation 2 and Formulation 3 to accelerated stress conditions to determine if the formulations would be stable and whether the problems of crystallisation at low temperatures would occur. The formulations were prepared in a concentration of about 6% w/v of cyromazine. The formulations were then subjected to a temperature condition of 4° C. for a period of 4 weeks and also at a temperature condition of 55° C. for a period of four weeks. As a positive control, a commercial formulation of cyromazine, sold under the brand name VETRAZIN®, and containing lactic acid, acetic acid and sulphuric acid was also tested. The results of this testing are shown below:

Analytical results for stressed samples of Cyromazine (about 6%)

| | | | pH at | % | % | % | % |
|---|---|---|---|---|---|---|---|
| Formulation | Temperature | | 20° C. | cyromazine | expected | LC | recovery |
| Cyromazine + | 4° | C. | 7.05 | 5.7 | 6.0 | 95.0 | 100.0 |
| PEG 200 | 55° | C. | 6.91 | 5.6 | 6.0 | 93.3 | 98.2 |
| to vol | | | | | | | |
| Cyromazine + | 4° | C. | 6.98 | 5.9 | 6.0 | 98.3 | 100.0 |
| 90% PEG | 55° | C. | 6.41 | 5.8 | 6.0 | 96.7 | 98.3 |
| 200 + 10% | | | | | | | |
| water | | | | | | | |
| Cyromazine + | 4° | C. | 6.87 | 6.0 | 6.0 | 101.7 | 100.0 |
| 80% PEG | 55° | C. | 6.23 | 6.0 | 6.0 | 100.0 | 98.4 |
| 200 + 20% | | | | | | | |
| water | | | | | | | |
| Cyromazine + | 4° | C. | | 5.85 | 6.0 | 97.5 | 100.0 |
| 40% PEG | 55° | C. | | 5.83 | 6.0 | 97.2 | 99.7 |
| 400 + 60% | | | | | | | |
| water | | | | | | | |
| Cyromazine + | 4° | C. | | 5.91 | 6.0 | 98.5 | 100.0 |
| 40% PEG | 55° | C. | | 5.93 | 6.0 | 98.8 | 100.3 |
| 400 + 60% | | | | | | | |
| water | | | | | | | |
| Vetrazin ® | 4° | C. | 6.94 | 6.2 | 6.0 | 103.3 | 100.0 |
| | 55° | C. | 6.95 | 6.1 | 6.0 | 101.7 | 98.4 |

Further long term stability tests for 12 months at room temperature (25° C.) were conducted on three sample formulations and these showed that the formulations were stable. The results of this testing are shown below:

| Formulation | % Cyromazine | % expected | % LC | % recovery |
|---|---|---|---|---|
| PEG 400 - 40% | 5.9 | 6.0 | 97.5 | 96.7 |
| Benzyl Alcohol - 1% (preservative) | | | | |
| Tween 80 - 1% (wetting agent) | | | | |
| PVP - 0.2% (polymer) | | | | |
| Acid Red 4R - 0.008% (colouring agent) | | | | |
| Water to 100% | | | | |
| PEG 400 - 40% | 5.9 | 6.0 | 98.5 | 96.7 |
| Benzyl Alcohol - 1% (preservative) | | | | |
| Tween 80 - 1% (wetting agent) | | | | |
| PVP - 0.2% (polymer) | | | | |
| Acid Red 4R - 0.008% (colouring agent) | | | | |
| Water to 100% | | | | |
| PEG 400 - 40% | 6.0 | 6.0 | 99.5 | 98.3 |
| Benzyl Alcohol - 1% (preservative) | | | | |
| Tween 80 - 1% (wetting agent) | | | | |
| PVP - 0.2% (polymer) | | | | |
| Acid Red 4R - 0.008% (colouring agent) | | | | |
| Water to 100% | | | | |

Accordingly, as a result of the formulation studies carried out by the Applicant, they have been able to produce a stable aqueous based formulation of cyromazine, by providing a solution of cyromazine in a solvent system containing water and PEG. It is envisaged that the solvent system could contain a single grade of PEG or two or more different grades of PEG if desired. If the amount of cyromazine is 6% w/v or more, the Applicant has found that the formulation is particularly stable when the total amount of PEG or PEGs in the formulation is at least 30% v/v.

The Applicant has found that the following commercially available grades of the PEGs are particularly suitable for use in the invention: PEG 200, PEG 400, PEG 1000, PEG 6000, and PEG 8000. However it will be noted that these grades are indicative of the average molecular weight of each grade, and thus it is better to express the range as the or the majority of the polyethylene glycols used in the formulation have an average molecular weight in the range from 200 to 35000.

The cyromazine is advantageously present in the formulation in an amount of from 0.1 to 20% w/v. If the amount of cyromazine is 6% w/v or more, PEG is advantageously present in the formulation in an amount of at least 40% (shown as % v/v in the case of the liquid PEGs, or shown as % w/v (in the case of the solid PEGs) if the formulation is prepared without heating.

The formulations of the present invention may include further excipients such as colouring agents, stabilizers, preservatives, buffers, thickeners, spreading agents, wetting agents and the like.

The formulations of the present invention may also include further active ingredients such as additional pesticides.

Advantageously, the formulations of the present invention are effective for the treatment, control or prevention of ecto-parasites in animals, particularly blow fly on sheep. Advantageously, the formulations are pour-on or spray-on formulations for topical administration.

Pour-on or spray-on formulations are generally administered in small volumes, for example, conventional dosing guns for administering a pour-on formulation would typically deliver about 50 ml quantities of a formulation. In the case of a sheep pour-on, the applicator guns are usually adapted to supply a dose of about 5 ml to 30 ml. The formulations of the present invention are typically designed to be delivered in a dosage amount of about 10 ml per 10 kg of live bodyweight of the animal to be treated. For example, a 50 ml dose of a pour-on formulation of the present invention would supply sufficient active to treat a 50 kg sheep.

Thus it can be seen that a stable aqueous based formulation of cyromazine, or a pharmaceutically acceptable salt thereof, has been provided which has several advantages, including: the provision of physically stable formulations which have a reduced tendency to crystallise at low temperatures and thereby cause product failure; the provision of safe aqueous formulations which are non toxic to users and have high tolerability on sheep, as well as easy application and dispersion through sheep's wool to reach the infected or targeted areas; the provision of a formulation which is easy to manufacture and cost-effective while still providing high efficacy against ecto-parasites such as blow fly. It is also possible to provide a liquid concentrate containing cyromazine, or a pharmaceutically acceptable salt thereof, which can be diluted with water.

Having thus descried in detail advantageous embodiments of the present invention, it is to be understood that the invention defined by the above paragraphs is not to be limited to particular details set forth in the above description as many apparent variations thereof are possible without departing from the spirit or scope of the present invention.

What is claimed is:

1. A liquid veterinary formulation comprising a stable solution of cyromazine, or a pharmaceutically acceptable salt thereof, in a solvent system containing water and a sufficient amount of one or more of the polyethylene glycols (either a single grade or a combination of two or more different grades) to keep the cyromazine in solution at ambient temperature.

2. A liquid formulation as claimed in claim 1 wherein the or the majority of the polyethylene glycols used in the formulation have an average molecular weight in the range from about 200 to about 35000.

3. A liquid formulation as claimed in claim 1, wherein the cyromazine, or a pharmaceutically acceptable salt thereof, is present in an amount of from about 0.1 to about 20% w/v.

4. A liquid formulation as claimed in claim 1, wherein the cyromazine, or a pharmaceutically acceptable salt thereof, is present in an amount of at least 6% w/v.

5. A liquid formulation as claimed in claim 1 wherein the total amount of PEG or PEGs in the formulation is from about 30% v/v to about 99% v/v.

6. A liquid formulation as claimed in claim 1 wherein the total amount of PEG or PEGs in the formulation is from about 40% v/v to about 90% v/v.

7. A liquid veterinary formulation comprising a stable solution of cyromazine, or a pharmaceutically acceptable salt thereof, in a solvent system containing water and one or more of the polyethylene glycols (either a single grade or a combination of two or more different grades), wherein the total amount of polyethylene glycol in the formulation is at least 30% v/v.

8. A liquid veterinary formulation as claimed in claim 7, wherein the cyromazine, or a pharmaceutically acceptable salt thereof, is present in an amount of at least 6% w/v.

9. A liquid concentrate capable of being diluted with water to make a stable aqueous solution of cyromazine, the concentrate comprising a stable solution of cyromazine, or a pharmaceutically acceptable salt thereof, in a liquid polyethylene glycol.

10. A liquid concentrate as claimed in claim 9, wherein the concentrate contains one or more PEGs having an average molecular weight in the range from about 200 to about 400.

11. A method of treating, controlling or preventing ectoparasites in an animal by administering to said animal a liquid veterinary formulation or a liquid concentrate diluted with water as claimed in claim 1, 7 or 9.

12. The method as claimed in claim 11, wherein the formulation is administered topically to said animal.
